(19) United States
(12) Patent Application Publication
(10) Pub. No.: US 2009/0216252 A1
(43) Pub. Date: Aug. 27, 2009

(54) A COUPLING DEVICE ENABLED BY MECHANICAL CONTINUITY OF CELLULAR SCAFFOLDING ACROSS TISSUE BOUNDARIES

(75) Inventors: David Boyd Melvin, Loveland, OH (US); Alan J. Melvin, Loveland, OH (US); Mark T. Byrne, Loveland, OH (US)

Correspondence Address: WOOD, HERRON & EVANS, LLP, 2700 CAREW TOWER, 441 VINE STREET, CINCINNATI, OH, 45202, US

(73) Assignee: THE UNIVERSITY OF CINCINNATI, Cincinnati, OH (US)

(21) Appl. No.: 11/722,726

(22) PCT Filed: Feb. 11, 2005
(86) PCT No.: PCT/US05/04679

§ 371 Date: Nov. 4, 2008

Related U.S. Application Data
(60) Provisional application No. 60/544,721, filed on Feb. 13, 2004.

(51) Int. Cl. A61B 17/08; A61B 17/04

(52) U.S. Cl. 606/151; 606/228

(57) ABSTRACT

A device and method for achieving load-bearing living-tissue-to-living-tissue coupling comprises a myriad of fine fibers extending directly from within the substance of one tissue to within the substance of the other tissue. Fibers are similar in cross-sectional area to, or smaller than, host tissue cells. This enables fibers to provide a scaffolding into which proliferating cells of each tissue may grow to form a collagenous matrix enveloping individual fibers and transferring mechanical loads between each tissue's extracellular matrix and the fibers. Also taught are devices and methods (1) for delivering bundles of independent fibers into soft or hard tissue, (2) for transiently reducing tissue drag during insertion, (3) for temporarily stabilizing position during tissue ingrowth, and (4) for spatial distribution of fiber bending stress in the event of a hard tissue.

### RELATED APPLICATIONS

This application claims priority from U.S. Provisional Patent Application No. 60/544,721, filed Feb. 13, 2004, which is incorporated herein by reference in its entirety.

### FIELD OF THE INVENTION

This invention relates broadly to systems and methods for coupling a tissue with other structures, such as another tissue, bone and prosthetic devices.

### BACKGROUND OF THE INVENTION

In the art and science of surgery, and in all its subspecialties, one basic requirement is a reliable and durable means of attaching or reattaching living tissue either to another living tissue or to a nonliving, or prosthetic, structure. Although durable and reliable direct tissue fusion technologies, such as by chemical (adhesive) or energy-infusion (welding) means, are intriguing, their practical application is thus far rather limited.

The remaining fasteners or devices now available are mechanical, namely, sutures and staples. These devices transfer tensile forces to and from the living tissue by an interlock mechanism, in which force-bearing surfaces are at a positive angle to the force direction, so that a directly compressive, or ‘normal’ force is exerted on and borne by the tissue being contacted. This creates pressure generally equal to the transmitted force divided by the projection of bearing area perpendicular to the force. There are important applications in which these fastening mechanisms often fail, because of small available bearing areas, tissues that can tolerate only mild pressure, or both. Examples of this are use of staples or sutures to repair slender tendons of a forcefully contracting muscle and closure of tissue which lack the strength granted by substantial amounts of organized collagen-such as brain, muscle, fat, or liver.

The use of polymers (degradable and non-degradable), natural fibers, metals and glass as filaments, as well as other geometries, for tissue culture scaffolds in outside-the-body (ex vivo) “bioreactors” are generally known. Many materials, both degradable and permanent, have been employed for well over two decades as substrates or scaffolds to provide a mechanical structure of desired geometry for cultured cells to adhere to and grow. Adhesion of cells to a scaffold material appears to be related to initial adherence, or ‘adsorption’ of tissue proteins, particularly fibronectin, to the material. Various polymers used for medical devices have been used as a tissue scaffold. Absorbable ones include polyglycolic acid and poly (lactide co-glycolide). Nonabsorbable polymers include polyurethanes, polyethylenes, polydimethylsiloxane, polysulfone, polymethyl methacrolate or pMMA, poly (2-hydroxymethylmethacrolate) or pHEMA, polypropylene, and polyvinyl chloride or PVC, among others.

No clear consensus exists as to a preferred composition or geometry. In a study of rat tendon fibroblast cells, growth was achieved on each of three fibrous polymeric materials tried, Dacron (polyester or poly[ethylene terephthalate]), Nylon, and polyethylene. It was also shown that more linearly oriented cell growth (although slower) occurs on fibers as compared to flat surfaces of similar polymers. Hepatocytes grew in a bioreactor based on spirally wound nonwoven (sheets of 13 micron fibers) polyester matrix. U.S. Pat. Nos. 6,140,039, 5,800,541 and 5,885,829 each teaches a 3-dimensional filamentous or fibrous matrix structure to culture fibrous tissue that can subsequently be implanted as tendon or ligament grafts. U.S. Pat. No. 5,885,829 describes a variety of synthetic meshes for dental pulp regeneration in vitro. Surveys of absorbable polymer meshes, nonabsorbable polymer meshes, and polymer sponges that have been used as cell culture scaffolds are available. U.S. Pat. Nos. 5,580,781 and 5,478,739 teach a 3-dimensional fibrous cell culture system that may be inoculated with stromal support materials, including fibroblasts, and other cellular elements in sequence, U.S. Pat. No. 5,516,681 specifically applies this to pancreatic cell culture.

Glass fibers functioned well for neuronal cell culture, in sizes of 0.4 micron. Glass fibers in minuscule fragments, such as might be inhaled in an industrial setting, were toxic in cell culture, but longer fibers were not, with problematic observations (cytotoxicity and macrophage dysfunction) confined to fragments 15 microns in length and less than 1.2 microns in diameter. A specialized glass (Bioglass®) was also successfully used as a cell culture substrate. Carbon fibers have also been successfully employed as a substrate for fibroblast culture. Additionally, metals have been used as a tissue scaffold. A 50-micron titanium mesh was successfully used for bone marrow cell culture, assessing in vitro bone formation. U.S. Pat. No. 4,983,184 teaches a metallic fiber, generally a titanium alloy less than 20 microns, for cell cultures to generate artificial and reinforcing soft tissue components. While these fibrous tissue scaffolds demonstrate the capacity of tissue cells and natural proteinaceous fibers generated by those cells to adhere to the surfaces of such non-living filaments, these devices and scaffolds have heretofore been restricted to a single tissue.

Many of these same materials have been used as inside-the-body (in vivo) connectors of one tissue to another in which coupling relies on interlocking mechanisms (meshes, tied loops, braids, etc.) for normal force transfer. Polymer fibers have been used in vascular grafts with rare clinical failure even after over 40 years of retaining arterial pressure, although weakening with time has been observed at points of stress concentration. Vascular grafts using extremely small (3 micron) fibers, suggesting better tissue incorporation, have also been performed. Polyester meshes, weaves, and braids are described in U.S. Pat. No. 6,612,977 as ‘slings’ for urethral suspension that support the urethra by extension into pelvic tissues. Braided polymer structures have been used for ligament replacement (U.S. Pat. Nos. 4,728,329 and 6,599,319). U.S. Pat. No. 5,769,864 teaches a mesh tissue implant, U.S. Pat. No. 5,004,474 is a woven tissue implant, and U.S. Pat. No. 4,932,972 is a braided, polymer artificial ligament. U.S. Pat. No. 4,863,471 is a system of interwoven textile tubes, and U.S. Pat. No. 4,795,466 is a system of concentric textile tubes, used as ligament replacements. U.S. Pat. No. 6,599,319 teaches a braided artificial tendon of thermatropic liquid crystal filaments, and U.S. Pat. No. 6,193,754 a woven artificial tendon made of polyester. U.S. Pat. No. 4,917,699 is specified as a 3-dimensionally braided artificial ligament with eyelets at each end for attachment to bone. U.S. Pat. Nos. 4,255,820, 4,483,023, and 4,187,558 are artificial ligaments comprised of fabric, the latter also including an elastomeric component. U.S. Pat. No. 4,484,722 is a fabric artificial ligament impregnated with an elastomer and covered by an absorbable fabric.

Solid polymer or metal structures, with or without fiber reinforcements, have been used as bone anchors to mechanically lock onto and into the cortex of long bones, as taught by U.S. Pat. Nos. 4,590,928 and 4,851,005. U.S. Pat. No. 6,517,579 teaches an anchoring apparatus that extends through the width of a bone to expand on the opposite side from entry, exerting a normal force on that surface of the bone. U.S. Pat. No. 5,380,334 teaches a bone anchor with barbs that expand upon insertion of a pushrod to wedge and grasp the interior of a bone drill hole, U.S. Pat. No. 5,716,358 is a bone screw with ‘micro-bumps’ that allow interlocking with bone substance. U.S. Pat. No. 5,601,558 is a soft tissue to bone anchor with barbs. Titanium mesh has been used as a soft tissue anchoring and as a bone implant, U.S. Pat. No. 6,312,473 teaches a variety of materials, such as graphitic carbon, polytetraflurethylene, and titanium, used as meshes which are fused to solid orthopedic implants. While many of the connectors discussed above are attempts to couple tissues to each other or to a prosthesis, such direct (‘normal’) force transfer creates, by necessity, an increase in tissue pressure which intrinsically limits the degree of force transmission tolerated by the tissue. Moreover, the stress-concentrating effects inherent in weaving, braiding, or other organization increases the likelihood of material fatigue failure during several years of cyclic loading.

Parallel bundles of fibers have been used as tension-transmitting members across body spaces from one tissue to another, such as across a joint space from one bone to another as artificial ligaments. The filamentous structure does encourage tissue ingrowth for reinforcement of the tension member itself, but tissue coupling at the fiber ends is still via conventional interlocking penetrating elements such as sutures, staples, barbs, plugs or screws.

For instance, both polyester (poly[ethylene terephthalate]) and carbon fibers have been used as simple parallel-fiber configurations for knee (anterior cruciate) ligament replacement with good tissue and collagen fiber ingrowth witnessed at 36 weeks in rabbits. Experimental polyester looped about tendon stump and calcaneal bone in sheep effectively generated a fibrous neotendon. Parallel polymeric and carbon fiber bone implants were less successful experimentally, although ingrowth did occur. U.S. Pat. No. 4,662,886 extends parallel fibers through the length of a ‘surgical element’, constrained in a braided sheathing which has gaps or windows at intervals to expose some area of the core fibers for tissue ingrowth. U.S. Pat. No. 3,987,497 teaches containment of a core of linear polymer oriented in the longitudinal axis of the prosthesis constrained within a sheath of nonoriented cross-linked polymer, with the structure intended to be fixed to bone and cartilage by suture or adhesives. U.S. Pat. No. 4,642,199 teaches a combination of low modulus and high modulus fibers which may be loosely twisted together to give nonlinear elastic behavior. U.S. Pat. No. 5,263,984 teaches an artificial ligament comprised of random, generally parallel, absorbable fibers such as collagen or elastin, held together at intervals by polysaccharide molecules. U.S. Pat. No. 5,049,155 is a ligament prosthesis comprised of multiple strands of 0.026 to 0.08 inch (650 to 2000 micron) diameter expanded poly(tetraflurethylene). U.S. Pat. No. 4,773,910 teaches an artificial ligament made of two strands, each comprised of several thousand 25 to 50 micron diameter polyolefin (such as polyethylene) filaments, and attachable to bone at the two ends by a button and a loop, respectively.

These tension members, however, do not provide a substantial passage of unrestrained parallel filaments into the substance of tissue so as to provide adequate surface interface areas. While multifilamentous structures are used to couple tissue, they include restraining mechanisms to restrict the dispersion of filaments by ingrowing tissue, thus inhibiting near complete surface contact between prosthetic material and tissue. Generally, restraint is achieved by braiding together the fibers themselves. The least restrictive appears to be U.S. Pat. No. 4,662,886, noted above for an embodiment as an artificial ligament. This also teaches an embodiment as a suture of central, generally parallel, fibers or filaments, but dispersion is limited to a braided sheath, configured to leave gaps or windows exposing the central fibers to tissue ingrowth in some areas.

Coatings and fillers have been previously used for easing the passage of fibers, such as braided sutures, through tissue. For instance, a water-soluble coating for polyglycolic acid braided absorbable sutures has been used. U.S. Pat. Nos. 5,621,050, 5,442,016 and 5,123,912 teach specific copolymers that coat or impregnate sutures and reduce drag through tissue. U.S. Pat. No. 4,983,180 teaches coating of braided sutures with sucrose fatty acid ester, stearic acid combined with various copolymers, beeswax, or paraffin wax. U.S. Pat. No. 4,461,298 describes silk sutures impregnated with hydrophobic thermoplastic elastomers and U.S. Pat. No. 4,362,162 teaches multifilament sutures of unspecified composition filled with a copolymer of poly (tetraflurethylene) and a second unspecified monomer. U.S. Pat. No. 4,027,676 teaches a series of compounds to coat an absorbable suture, while U.S. Pat. No. 4,043,344 teaches a means of lubricating nonabsorbable sutures by coating the suture with a solid ‘pasty’ film made of polyoxyetheline and polyoxypropylene, which is absorbed in less than 48 hours when implanted. U.S. Pat. Nos. 4,590,928 and 4,851,005 describe the filling of bundles of carbon fibers, to be used as ligament replacements, with absorbable cross-linked gelatin before implantation. These coatings present absorbable lubrication or an absorbable smoothing filling for the intent of easing passage of the fibers through tissue.

Macro-textured surfaces have been used to improve tissue adhesion to implanted devices with extensions or cavities. U.S. Pat. No. 5,571,182 utilizes individually textured fine particles, injected through hypodermic needles, as a tissue filler. U.S. Pat. No. 4,955,907 teaches a fine texturing for inflatable, tissue-expanding implants. U.S. Pat. Nos. 4,846,834 and 5,011,494 teach a surface with both depressions and extensions, none described as exceeding 1 mm in length. U.S. Pat. No. 4,278,623 teaches a method of fragmenting ultrafine polymer fibrils so that they may be used to ‘flock’ or texture the surface of an implantable device. U.S. Pat. No. 6,083,244 and U.S. Patent Application 2003/0088270 teach a tendon repair device, which includes extensions, such as burrs, directed generally at right angles to the direction of force. While these surfaces provide increased interface area, the dimensions are generally too small to significantly increase tissue adhesion. The projections in previous applications, insofar as dimensions are specified, are at most 1 mm (1000 microns) or less in length/depth.

Micro-texturing of prostheses has also been used to augment tissue adhesion. For example, U.S. Pat. No. 4,522,596 teaches roughening the surface of titanium, vitalium, ceramic compositions or other biocompatible materials for the purpose of tissue adherence after bone implantation. While these are means to increase the static coefficient of friction of a surface, they have heretofore not been applied to fibers or filaments.

Hooks and barbs have also been incorporated as components of prior tissue fasteners. U.S. Pat. No. 4,562,596 teaches barbed hooks extending from the ends of an intraluminal aortic graft device to securely attach to the inside of the aorta. A “harpoon-shaped hook” on a metal wire suture for the repair of knee ligaments has also been used. A ‘Jenning's barb-wire’ has been used for thumb ligament repair. U.S. Pat. No. 4,534,352 teaches a ratchet-like resorbable polymer surgical fastener in which one member pushes through tissue into a receiving member and locks by snapping a barb through a hole in the receiving member.

While there has been significant progress made in the coupling of tissue with other structures, as discussed above, present devices and methods have some drawbacks which can be improved upon. As such, there is a need for a coupling device that addresses these and other drawbacks.

### SUMMARY OF THE INVENTION

According to the present invention, a device and method for achieving high load-bearing living tissue-to-living tissue coupling comprises a myriad of fine fibers extending directly from within the substance of one tissue to within the substance of another tissue, and an implanted prosthesis or coupling device. The fibers are similar in cross-section to tissue cells, or smaller, and provide a scaffolding into which proliferating cells of each tissue may grow to form a collagenous matrix enveloping individual fibers and transferring mechanical loads between each tissue's extra cellular matrix and the fibers. Also taught are: (a) a variety of suitable specific filament materials; (b) devices and methods for distributing bundles of filaments within tissue; (c) devices and methods for organizing filaments as they exit tissue, which may be of use in some applications; (d) devices and methods for reducing resistance, or ‘drag’, during initial surgical passage of bundles; (e) devices and methods for maintaining general spatial relationship of the coupling device to tissue during cellular and fibrillar ingrowth; (f) devices and methods for altering surfaces of filaments for the purpose of increasing coefficients of friction; and (g) devices and methods for lessening stress concentrations at exit points from hard tissues such as bone or tooth.

The features and objectives of the invention will become more readily apparent from the following Detailed Description taken in conjunction with the accompanying drawings.

### BRIEF DESCRIPTION OF FIGURES

The accompanying drawings, which are incorporated in and constitute a part of this specification, illustrate embodiments of the invention and, together with a general description of the invention given above, and the detailed description given below, serve to explain the invention.

FIGS. 1A-1C show an embodiment of a closure device for coupling tissue with another structure;

FIGS. 2A-2B show an embodiment of a closure device applied to a rectus abdominis muscle;

FIG. 3 shows an embodiment of a closure device similar to that shown in FIG. 1A but having needles on both ends;

FIGS. 4A and 4B show an embodiment of a closure device applied to a uterus;

FIGS. 5A and 5B show an embodiment of a closure device applied to the heart;

FIGS. 6A-6C show an embodiment of a closure device applied to a heart valve;

FIGS. 7A and 7B shows an embodiment of a closure device applied to bone;

FIG. 8 shows an embodiment of a closure device similar to

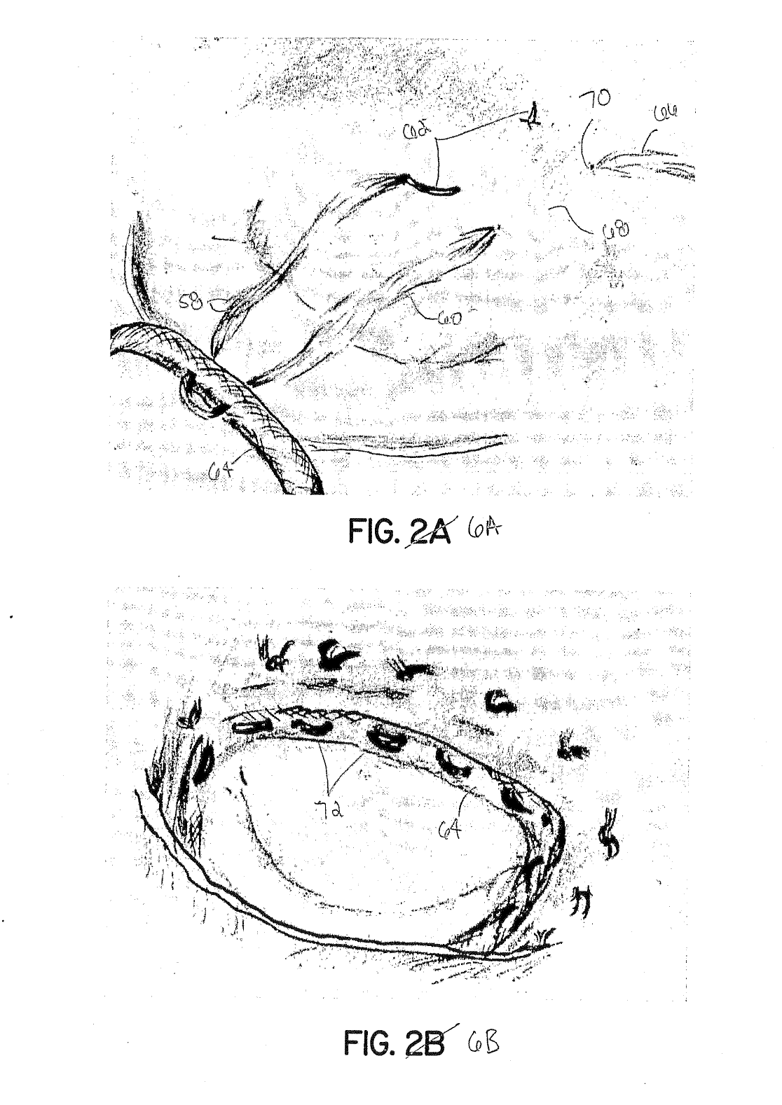

FIGS. 7A and 7B but having an absorbable or nonabsorbable soft plug;

FIG. 9 shows an embodiment of a closure device similar to

FIGS. 7A and 7B but having particulate material for inducing bone resorption;

FIGS. 10A-10C show an embodiment of a closure device having a multi-laminate patch attached to the surface of a bone;

FIGS. 11A and 11B show an embodiment of a closure device having a coil stress-distributing member;

FIGS. 12A and 12B show an embodiment of a closure device having a stabilizing clip;

FIG. 13 shows an embodiment of a closure device having a suture securing an exiting fiber bundle near the exit point;

FIG. 14 shows an embodiment of a closure device similar to FIG. 13 but having a patch;

FIGS. 15A and 15B show an embodiment of a closure device having a barbed strand nested among the fiber bundle;

FIGS. 16A-16C shows a slider for use with a closure device of the invention;

FIGS. 17A-17C show an embodiment of a closure device having a rigid absorbable filler with barb-like extensions;

FIGS. 18A-18D show an embodiment of a closure device having a double-ended rigid coupler; and

FIG. 19 shows an embodiment of a closure device having enhanced fiber dispersement.

### DETAILED DESCRIPTION

The invention employs a myriad of very fine continuous filaments that serve both (1) as substrate (or ‘scaffold’) for new tissue formation within the substance of one or more existing living tissues, and (2) as tension members holding that existing living tissue either to another living tissue or to a prosthesis. The invention employs the substantial tissue-prosthetic interface areas that are useful in tissue culture “bioreactors” (customarily ex vivo, or outside the body, in tightly controlled environments), to achieve durable bonding with one or more living tissues that are, instead, in vivo (inside the body). More particularly, the invention uses a multiplicity of bundles of filaments, drawn through a region of the living tissue. This creates, of each individual bundle of filaments, an effective slender bioreactor extending longitudinally within part of the substance of those living tissues. This allows adjacent cellular elements, particularly fibroblasts, to ingrow and adhere, in the complete milieu of surrounding tissue fluid factors.

In turn, these cells generate protein fibers (such as collagen), which strongly mechanically couple both to the filaments and to the physical framework of the tissue itself, its ‘extracellular matrix’. Each individual device filament extends, without interruption, beyond the boundaries of the living tissue either to another tissue (where they achieve similar bonding) or to an artificial or prosthetic structure where established mechanical attachments are employed. Since the biologically-induced protein fibers themselves will become a multitude of effective tiny tension members, tissue-to-tissue coupling may employ either degradable (temporary) or non-degradable (essentially permanent) filament material. On the other hand, no such biological fiber induction is to be expected in artificial implanted materials. Accordingly, for tissue-to-prosthesis coupling, non-absorbable and non-degradable filament material will generally be required.

In this way, force is transferred predominantly by shear stress, where the load capacity is determined by the product of the interface area, the tissue pressure, and the coefficient of friction at the interface. Remarkably large interface areas are achievable. The theoretical potential is the number of filaments times their circumference times their average length, and experiments have demonstrated near complete actual tissue contact by rapid tissue element ingrowth. The extensive area allows substantial bonding strength without substantial elevation of tissue pressures above normal pre-implant values. The counterforce induced in tissue must still, for equilibrium, equal the potential disrupting force. However, dividing this shear force by the large (relative to that available for normal-force coupling) bearing area yields shear stresses that are significantly smaller than the compressive stresses seen in normal-force coupling. The large number of filaments over which transmitted force is distributed, the continuity without inter-material coupling across tissue boundaries, and the minimization of bending stresses due to extremely small ratio of filament radius to any likely bending radius all combine to lessen the likelihood of tensile element fatigue failure.

The invention also describes a variety of suitable specific filament materials, means of distributing bundles of filaments within tissue, means of organizing filaments upon tissue exit that may be of use in some applications, means and materials to reduce resistance, or ‘drag’, during initial surgical passage of bundles, means and materials for temporarily stabilizing a position during tissue ingrowth, and for spatial distribution of fiber bending stresses in the event of a hard tissue.

The present invention achieves effective load-bearing coupling between tissue-and-tissue or tissue-and-device based on various different factors, including the observed existence of the following:

1. A finite positive pressure in most mammalian living tissues, at most times, of at least 5 mmHg (700 Pascals), and often 15 mmHg (2000 Pascals) above ambient atmospheric pressure;

2. Finite coefficients of kinetic and static friction between most living tissues and most prosthetic materials (even many of the materials selected for the opposite goal of low friction, e.g., artificial joint-bearing surfaces) of at least 0.02 and often 0.1;

3. A network of organized collagen (a specialized crystalline fibrous protein which, particularly in the structurally important type I molecular configuration, has substantial tensile strength), called the ‘extracellular matrix’, enveloping the living cells of almost all mammalian tissues. This collagen network, though often loose and accounting for only a small portion of tissue mass, accounts for the majority of tensile strength for most non-calcified tissues, either those not primarily constructed of collagen (e.g., brain, muscle, fat, kidney, liver, pancreas, etc.) or those that are (e.g., ligament, tendon, fascia, dermis, etc.); and

4. The tendency of most mammalian tissues to react to any type of implanted nonliving material, over a variable period of time, by generating an enveloping or encasing ‘capsule’ of collagenous tissue. The capsules formed about smaller caliber nonliving structure tend to be thinner and better supplied with blood vessels than are the capsules formed about larger ones. Data appears to indicate that this enveloping fibrous tissue, which may be termed an ‘extraprosthetic’ matrix, mechanically links, directly or indirectly, with the extracellular matrix.

The reaction of tissue to the fibers of the invention to facilitate force transfer is apparently in a sequence: (a) migration of inflammatory cells and other cells between adjacent fibers to separate them, followed by (b) ingrowth of fibroblasts and/or differentiation of other cells into fibroblasts to progressively displace inflammatory cells, with synthesis of collagen and/or elastin fibers that are directly or indirectly coupled mechanically to the extracellular matrix of the surrounding tissue. This linkage, combined with the shear force sustainable over the extensive surface area due to existing tissue pressure and almost any finite interface coefficient of friction, even if very small (e.g., 0.01 to 0.10), produces an effective load-bearing mechanical bond between the fibers and the tissue.

That such bundles, so inserted in tissue, will, in fact, achieve the theoretically computed interface area and mechanical load transfer is supported by evidence documented in four separate experimental models in three species in three investigational institutions: two studies of rabbit posterior tibial muscle, canine latissimus dorsi muscle and two completed studies in alternate animal models. This evidence is based both on direct tensile tests and tissue microscopy.

In one embodiment of the invention, the system includes multiple flexible longitudinal prosthetic elements, or fibrils, each of which has a cross-sectional area within two orders of magnitude of (i.e., between 0.01 and 100 times) the nominal or average cell cross-sectional area of a particular tissue, or from approximately 0.25 to approximately 30,000 square microns (uM²). In an embodiment where the fibrils are of circular cross-section (given only as a nonlimiting example of possible fibril shape), this would be about equivalent to a diameter range of 0.5 to 200 microns or 0.0005 to 0.2 millimeters.

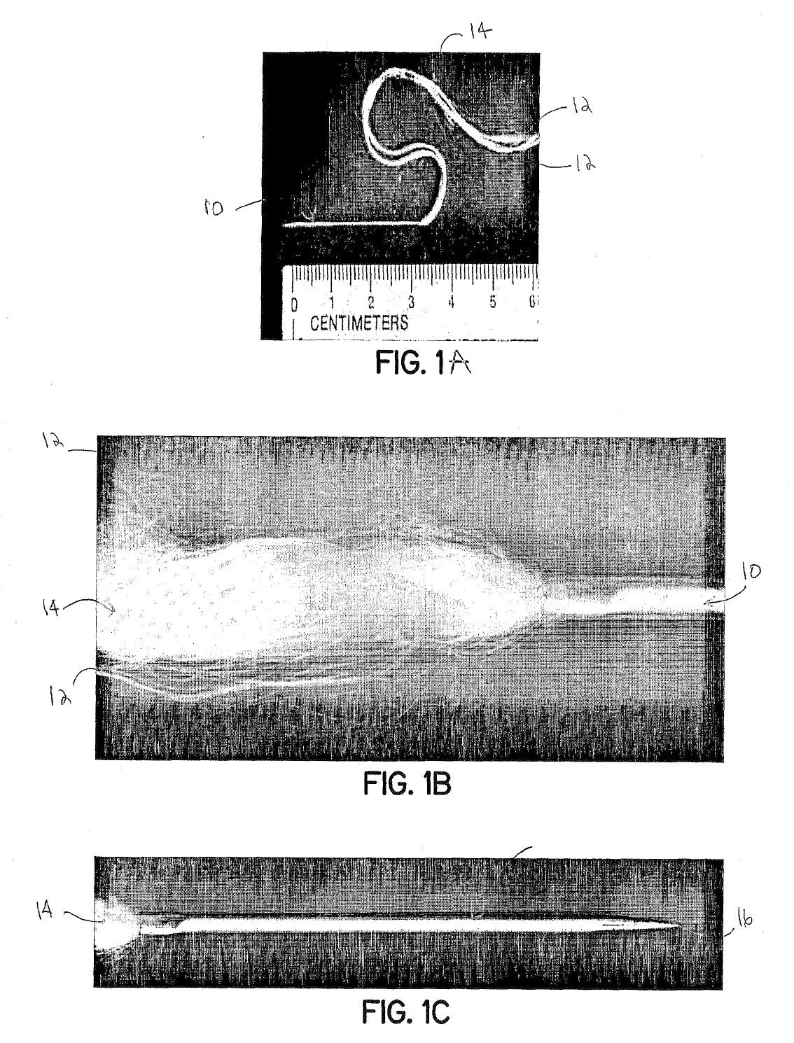

A device is used for inserting these filaments in groups, or ‘bundles’, of a few hundred to several thousand fibrils each, into the substance of soft or hard tissue. In one embodiment, this may include swaging each bundle into the heel of, or otherwise attaching to, one or more surgical needles of a type (curved, straight, taper-point, ball-point, cutting point, and so forth) commonly used to insert ordinary monofilamentous or braided sutures. Referring to FIG. 1A, a surgical needle 10 is illustrated, having a plurality of fine fibers 12 that collectively define a fiber bundle 14, which is swaged into the non-sharp end or heel of the surgical needle 10. FIG. 1B is an enlarged view of the fiber bundle 14 illustrating a relatively large number of fibers 12 extending from heel. For instance, approximately 2250 fibers that are each approximately 12 microns in diameter, may be swaged to the heel of a surgical needle 10. Those of ordinary skill in the art will recognize that fewer or additional fibers 12 may be used depending on the particular application. FIG. 1C is an enlarged view of the surgical needle 10, which includes a sharpened end 16 for facilitating insertion of the surgical needle 10 and fiber bundle 14 into hard or soft tissue.

In an alternative embodiment of the invention, the end of the fiber bundle 14 that is joined to tissue may be made relatively rigid, such as by locally impregnating the fibers 12 with a filler (absorbable or nonabsorbable) or by fusing fibers 12 with heat or chemical solvents in a desired region. In yet another alternative embodiment of the invention, the fiber bundle 14 may contain a relatively rigid sharpened elongate structure within it and be adhered to that structure by one or more absorbable ligatures and/or by one or more absorbable adhesive compounds, with the elongate structure comprising an absorbable needle. This absorbable needle may or may not have barbs or hooks to aid in position stabilization as shown in FIGS. 17A, 17B, 17C, 18A, 18B, 18C, and 18D.

Various methods and devices may be used for inserting fiber bundle 14 into tissue, and holding or positioning tissue and fibers, their ends, and/or their needles to facilitate a desired pattern of path of insertion in the tissue. For example, any of the methods taught in U.S. Pat. No. 6,733,510, which is incorporated by reference herein in its entirety, may be used. Thus, mechanical sliding mechanisms, pneumatic, hydraulic, or spring-acting ‘harpoon’ type mechanisms, the numerous other mechanisms described therein, and any or all of the art taught in that application may be used for inserting the embodiments of the present invention.

In one embodiment, a pattern of organization of fibrils in tissue such that between 200 and 40,000 cm² of prosthetic (filament) surface area are presented per cm³ of implanted prosthetic material volume. These ratios are computed for a nonlimiting example of a circular cross-section fiber, with the extreme examples of 200 and 40,000 applying to fibers of 200 microns and 1 micron (0.000,2 to 0.000,001 meters), respectively. To illustrate this example, a block of tissue of 4 cm×5 cm, or 20 cm², cut surface, held either to another similar or dissimilar tissue structure or to a prosthesis by a coupling device composed of 10 micron diameter (circular cross-section) fibrils displacing just 5% of that cross-section (i.e., tissue cross-sections subjacent to and parallel to the surface being coupled), and yielding a cumulative single-fibril cross-sectional area of about 1.2 mm² over an average depth of 4 cm (measured from the surface into the tissue), would have a total tissue/prosthetic material interface of 16,000 cm², or 1.6 m². If filaments displaced only 1% of tissue (i.e., 0.2 cm² of fiber cross-section for the 20 cm² of tissue cross-section, or, equivalently, 0.8 cm³ of fiber volume within the 80 cm³ tissue volume within 4 cm of that surface), the potential interface surface would be 3200 cm², or 0.32 m².

In the first example given above, (5% displacement of tissue for a 4 cm depth by fully dispersed 10 micron diameter filaments), assuming a 0.1 coefficient of static friction and 15 mmHg (i.e., ˜2000 N/m²) interstitial pressure in the tissue, a separation force of 320 N, or about 75 pounds, would be sustained by the shear loading of the interface. Clearly, the tensile strength of the material must be such that the approximately 1 cm² cumulative filament cross-section would sustain that 320 N load. In accordance with one aspect of the invention, this tensile strength (3.2 MPa) is exceeded, many fold, by any of the materials set forth herein as examples.

In accordance with another aspect of the invention, a method of placement for the invention tissue coupling devices includes delivery into tissue such that the primary load-accepting surfaces of the device are aligned parallel to (that is, at a zero or very small angle to) the existing or potential separating force, rather than aligned perpendicularly to that separating force. This is distinguished from existing sutures and staples in which the load-accepting, or load-bearing surfaces, whether the body of the suture or staple or projections such as barbs or hooks from that body, are aligned at a substantial angle (generally at least 15 degrees when bearing loads) to the separating force. The present invention presents a filamentous (that is, fibrillar or fibrous) coupling device configuration which has potential tissue/prosthetic interface contact area between one and three orders of magnitude (10 to 1000 times) the coupled tissue region's cross-sectional area in any plane perpendicular to the potential separating force. The invention also provides a method of placement of a coupling device which facilitates actual realization of tissue/prosthetic interface contact area between one and three orders of magnitude (10 to 1000 times) the coupled tissue region's cross-sectional area in any plane perpendicular to the potential separating force in a relatively short period of time (generally between seven and seventy days) after implantation.

The prosthetic material fibrils may be of a synthetic suitable material, including, for example, polymer, such as polyester, polypropylene, or polyethylene (especially linearly crystalline, high strength polyethylene); metal, such as stainless steel, titanium, titanium alloy, cobalt-chromium alloy, or nickel-titanium alloy (e.g., Nitinol®); natural animal or plant fibers such as silk, or wool, (with or without chemical modification or reconstitution); fibers of reconstituted animal or plant materials such as collagen, fibronectin, elastin, cellulose, or cross-linked gelatin; or other material such as fused silicon dioxide or other glass. Part of the rational for metals is that some, particularly steels and pure titanium, have established fatigue limits, i.e., levels of stress below which there is no fatigue failure regardless of number of cycles of cyclic stress. Bundles or devices may contain more than one type of fiber and individual fibers may contain more than one material, as, in a non-limiting example, a core chosen for tensile properties (elasticity, strength, etc) and one or more layers of surface coatings added to modify physical, chemical, or biologic interaction with adjacent living tissue.

In accordance with other aspects of the invention, several different embodiments may be used for different couplings. For example, devices may be employed to couple one tissue to another similar or dissimilar tissue. Specifically, the tissue may be two soft tissues (nonlimiting examples of many being skeletal muscle, uterine muscle, smooth muscle, cardiac muscle, loose connective tissue, dense connective tissue such as tendon or ligament, fat, brain). Alternatively, two hard tissues (e.g., bone, teeth) might be coupled, or soft tissue might be coupled to hard tissue.

The purpose for coupling may be for a variety of reasons including re-approximation of previously joined tissues after purposeful surgical incision; re-approximation of previously joined tissues after traumatic disruptions, whether by blunt or sharp trauma; approximation of tissue to close defects left by excision of diseased tissue, such as cancerous or inflammatory disease; and/or re-arrangement of tissue for functional (one nonlimiting example of many being tendon transfers for neurologic dysfunction), cosmetic (one nonlimiting example of many being attachment of a forehead flap during nose reconstruction), or other purpose, to name a few.

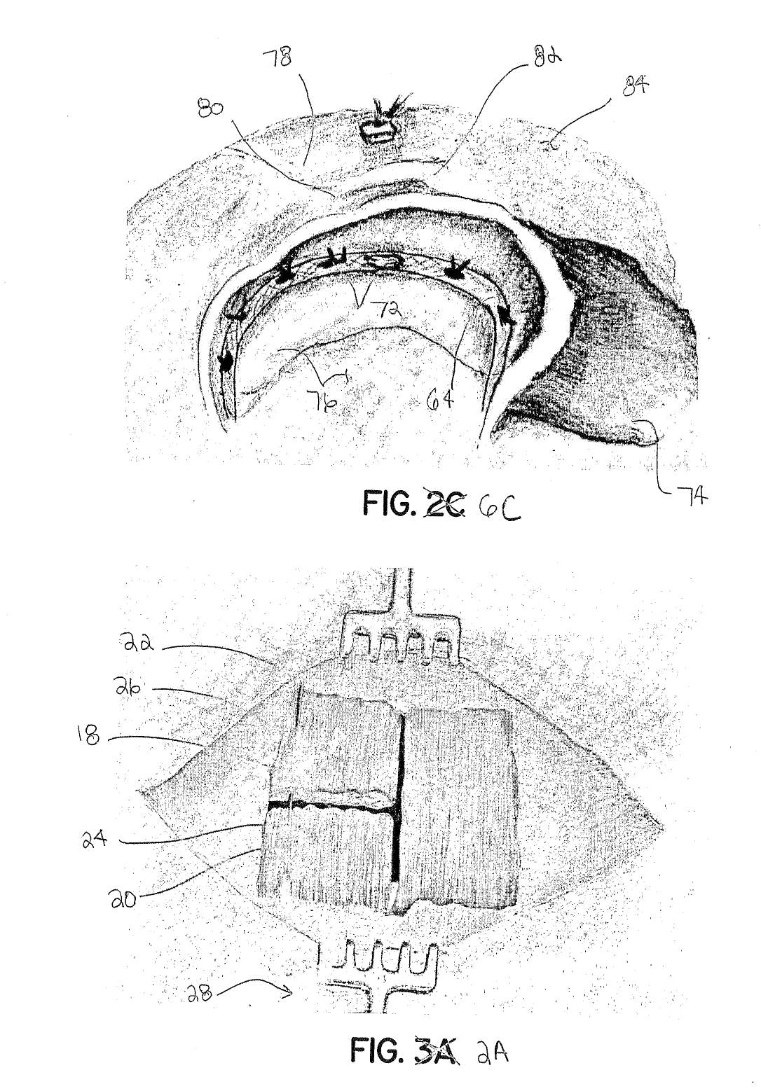

The following Figures illustrate various uses of the invention for coupling tissue. For example, repair of skeletal muscle after trauma or surgical incision in the absence of adequate organized fibrous tissue for conventional sutures may be done and is shown in FIGS. 2A and 2B. Turning to FIG. 2A, a transverse incision 18 is shown through a rectus abdominis muscle 20. The tissue normally relied upon for closure strength, that is, the anterior rectus fascia and linea alba, are missing. In accordance with one aspect of the present invention, a straight needle 22 is inserted in the lower part 24 of the muscle 20 and advanced across the incision 18 into the upper part 26 of the muscle 20, pulling the fiber bundle 28 behind it. With such a coupling, and the dispersal of tissue around and through the fiber bundle 28, the two parts of the muscle 20 may be coupled together.

FIG. 2B shows multiple closure devices utilized in accordance with the present invention. The closure devices 30 are shown securing the incision 18 in the right rectus muscle 20 (left side of FIG. 2B), together. The insertion of the fiber bundle 28 is done initially in the drawing of FIG. 2B in a direction opposite to that portrayed in FIG. 2A. Once passing through the muscle parts 24, 26 in one direction, the needle 22 is reversed, and the fiber bundle 28 is inserted in the opposite direction, followed by a tying of the suture in the upper part 26 of the muscle 20, as illustrated. For the purposes of illustration, the left rectus muscle (right side of FIG. 2B) shows a conventional type of closure 32 utilizing figure-of-8 sutures as is known in the art.

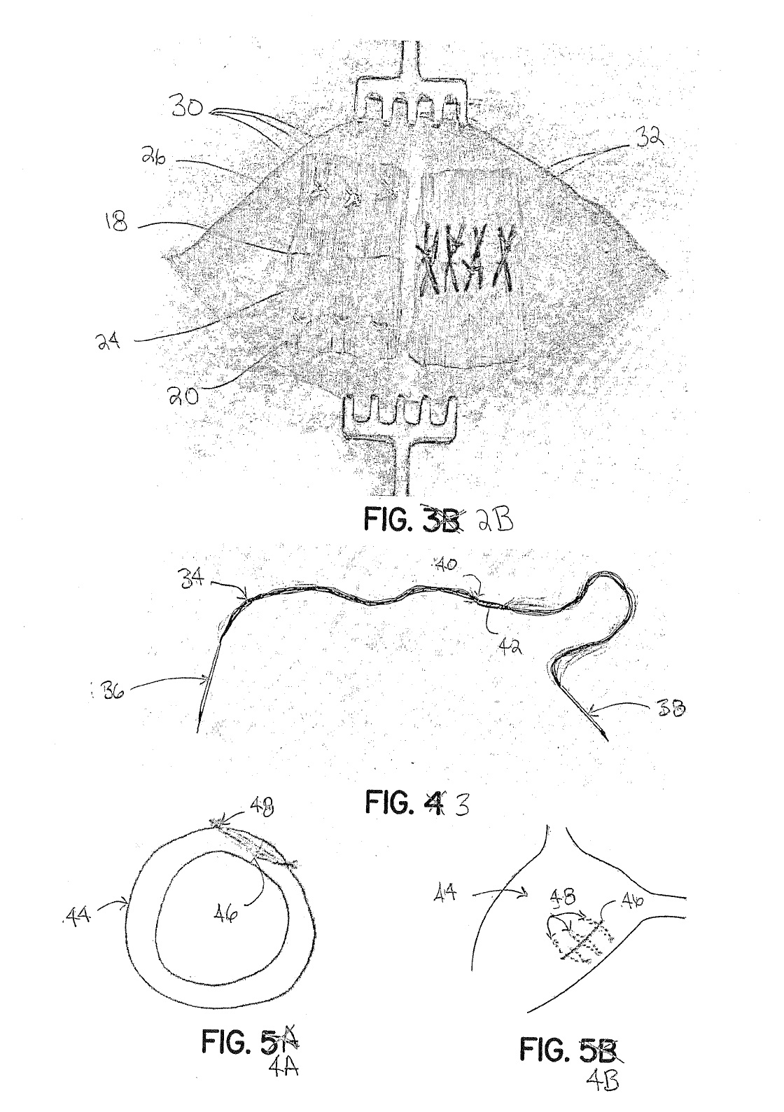

As an alternative to inserting the needle 22, reversing it, and then inserting it in an opposite direction, an alternative embodiment of the invention, as illustrated in FIG. 3, might be utilized wherein a fiber bundle 34 has needles 36 and 38 swaged onto each end of the fiber bundle 34. A short region 40 near the center of the closure device may be either braided, or may be surrounded by a tubular segment 42 of braided fibers, as shown in FIG. 3.

FIGS. 4A and 4B illustrate repair of uteri after fibromyotomy or Cesarean section. For example, FIG. 4A is a cross-section of a uterus 44, in which an incision 46 has been closed by a fiber coupling device 48 in accordance with the present invention, and as shown in FIGS. 1A-1C. FIG. 4B, on the other hand, is an external perspective view of the uterus 44, repaired in accordance with the present invention.

Alternatively, cardiac repairs may be made, as well. Referring to FIGS. 5A and 5B, a repair of a cardiac chamber 50 may be made after an incision or excision is made for any purpose. FIG. 5A illustrates a cardiac left ventricular cross-section in which an incision 52 has been closed by a fiber-coupling device 54 in accordance with the present invention. FIG. 5B is an external perspective view of the ventricle shown in FIG. 5A, illustrating the fiber bundle 56 passing through the heart wall adjacent the incision 52, as illustrated in FIG. 5A.

Similarly, other methods of utilizing the present invention might depend upon the tissue or part of the body in which the incision has been made. For example, fatty (adipose) tissue may be repaired after trauma or incision. Attachment or reattachment of muscle or tendon to a bone may be made after trauma, or for the purposes of reparative/reconstructive/cosmetic operations. Other methodologies of the present invention may include repair of the internal organs, such as the stomach, spleen, liver, kidney, lung, or intestine after extensive resection for any reason including, but not limited to malignant disease or after trauma of some kind. Furthermore, treatment of urinary stress incontinence, tendon transfer operations, tendon lengthening operations, and attachment or reattachment of muscle to tendon are also anticipated methods of utilizing the inventive device in accordance with the principles of the invention. Closure of dermis to dermis may also be accomplished with the invention when tension exceeds that permitted by conventional skin or subcuticular sutures or staples. Furthermore, reimplantation of teeth into mandible or maxilla might utilize the inventive device.

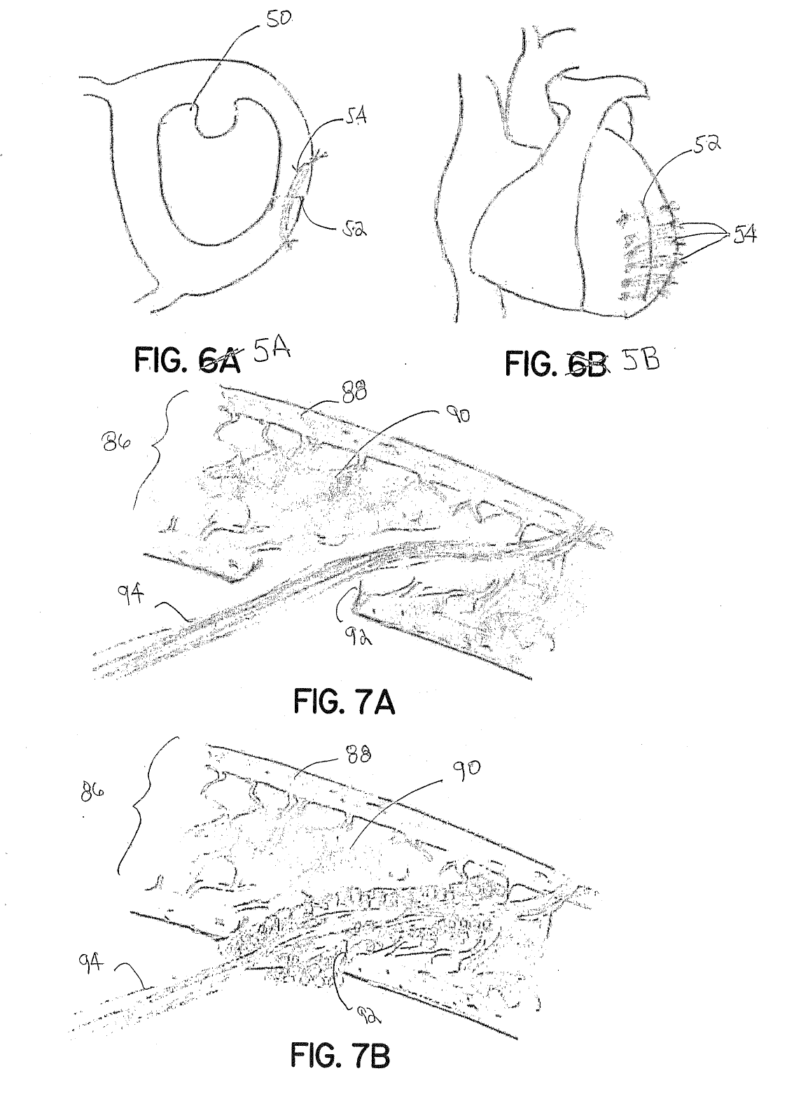

Alternatively, the invention may be employed to couple any tissue, hard or soft, to a nonliving (prosthetic) structure that is implanted in the body. For example, as illustrated in FIGS. 6A-6C, heart valve prostheses, such as replacement valve sewing rings, or annuloplasty rings, might be coupled to atrial (See FIGS. 6A and 6B), or ventricular (FIG. 6C) muscle. Turning to FIG. 6A, fibers 58 defining fiber bundle 60 are shown with a needle 62 in accordance with the invention, both ends having been placed through annuloplasty ring 64. One end 66 has been passed through the mitral annulus 68, and then tangentially through atrial wall 70. The needle 62 of the second end is also placed through the atrial wall 70. FIG. 6B illustrates multiple of the inventive coupling devices 72, placed and tied circumferentially around the ring 64. FIG. 6C shows a cut-away view through the left atrium 74, wherein an annuloplasty ring 64 is alternatively secured. FIG. 6C illustrates the fiber bundle 60 of the coupling device 72 of the invention that has been placed through an annuloplasty ring 64 and through the annulus of the mitral valve 76. Then, the bundle 60 is passed downward through the basal ventricular wall 78, deep to the circumflex coronary artery 80 and coronary sinus 82, to exit on the ventricular surface 84, where it is tied over a pledget (of Teflon® felt as shown, or alternatively of pericardium or other available tissue).

In another alternative coupling, in accordance with the present invention, skeletal muscles, tendons, or ligaments may be coupled to prosthetic bones. Similarly, skeletal muscles, tendons, or ligaments might be coupled to prosthetic connective tissue, which for nonlimiting examples are a nonabsorbable and absorbable polymer mesh. Skeletal muscles, tendons, or ligaments might also be coupled to prosthetic joints. In another alternative embodiment, skeletal muscles may be used as power transmission devices which, in turn, drive cardiac or other power-consuming power devices. The present invention might also be utilized to anchor dental prostheses to a mandible or maxilla.

In accordance with an alternative embodiment of the invention, the surfaces of the fibers might be modified. In that way, the coefficient of friction might be increased, and thus shear stress/normal stress ratio increased. The limit of safety is the point at which either normal or shear stresses exceed biologic tolerances by either mechanical disruption or reduction of perfusion. With existing coefficients of friction, without surface modification (e.g., 0.02 to 0.10), it is likely that normal stresses are the critical limiting factor for biologic intolerance, whereas it is the shear stresses that determine holding effectiveness. The ratio of benefit (holding effectiveness) to risk (tissue damage or perfusion compromise) could be increased by increasing the relative coefficient of friction. This may be done in several ways in accordance with different embodiments of the invention.

For example, sintering might be used, including fusing minuscule (e.g., 500 ng to 2 micrograms greatest dimension) particles, spherical or cubic or other shaped, to surfaces of fibers by chemical (e.g. solvent bonding) or thermal (e.g. welding) or other process. Also, mechanically pitting of the fibers by a variant of sand-blasting (air- or gas-propelled abrasive particles) might be used. Mechanical pitting might also be accomplished by placing fibers in a bath of suspended fine metallic or ceramic or other grit particles and subjecting them to ultrasound. Other pitting techniques might also be used, such as pitting by electron or other subatomic particle bombardment, pitting by laser (ultraviolet, infra-red, microwave, visible light, or other) and pitting by gamma, x-ray, or other radiation.

Surface modification might also be accomplished by adhering a surface coating to fibers with another biocompatible material, such as a polymer, metal, or ceramic. The coating could be optimally selected for frictional effects while the core fibers would be optimally selected for mechanical characteristics such as strength, elastic behavior, and flexibility. Coatings may be applied to the fibers using various techniques including solution casting, vapor deposition, or particle ‘sputtering’ deposition techniques.

Alternatively, purposefully entangling and/or focal fusing between individual fibers in ‘minibundles’ of a few (e.g., 2 to 100) individual fibers could be used. Many of these minibundles could then be grouped into bundles to allow practical placement into tissue by techniques already established. In that way, the shear force holding would be supplemented by normal forces of a myriad of minuscule loops encircling, and being encircled by, solid tissue structures such as collagen or muscle fibers. Controlled heating could also be used to fuse or weld fibers at random contact/crossing points in a fiber bundle. Alternatively, controlled surface solvent exposure by immersion or misting might also be done.

Adhesion prevention techniques might also be used in the inventive device as an alternative to the fiber-reinforced polymer sheets taught in patented muscle coupling applications, such as shown in U.S. Pat. No. 6,214,047. Such adhesion prevention techniques will generally be useful for tissue-to-prosthetic applications somewhat similar to the application envisioned in that earlier patent, to protect exposed fiber from adherence to surrounding tissue, restricting motion. Such means and methods may be useful in tissue-to-tissue applications of the more generally applicable invention taught herein. A solution cast polymer membrane is currently used, an aliphatic polyurethane (Tecoflex® solution-cast from methylene chloride solution and incorporating a thin Nylon® mesh).

In one embodiment, a layer of Seprafilm bioresorbable membrane (Genzyme Corp.) might be used, or a thin membrane of expanded polytetraflurethyiene (ePTFE) such as that marketed as Gortex® or Impra®. A topically applied anti-adhesive compound, CoSeal®, marketed by Baxter Healthcare, Inc., might also be used. One or more sheets of silicone sheeting with polymer mesh reinforcement marketed by several suppliers under license from Dow Corning could also be used. Still further, alternative mesh-reinforced polymers with alternative polymer mesh (such as polyethylene, polypropylene, or poly (ethylene terephthalate) instead of nylon, and alternative solution-castable elastomer (such as solvent-suspended polydimethylsiloxane or silicone rubber) might be suitable for adhesion prevention.

In another embodiment of the invention, alternative means of unraveling prevention, i.e., of holding core parallel fibers into a compact bundle is used (still minimizing stress concentration) in any extensive region not traversing tissue to which bonding is desired. An all-parallel fiber bundle would be an improvement because the external braid of the kernmantel configuration as presently used diverts a portion of available fibers from direct, most effective load-bearing in the cord formed at an exit from muscle. Furthermore, the braid adds more diameter to the cord than may be possible with alternatives.

A polymer anti-adhesive sheath may be used for this function as well. Alternatively, impregnating or coating an all-parallel bundle with an elastomer may be used. Stress concentration potential at the boundary between impregnated and unimpregnated regions could be minimized by graded increase in durometer/stiffness of the elastomer beginning with a very low durometer/stiffness at fiber entry, graded decrease in porosity of the elastomer beginning with a very low porosity at fiber entry, and branching of the impregnated elastomer with separating fiber bundles into the most distal muscle before discontinuance.

In another alternative, encasing the combined bundles in one or more groups, compacted within a braided, woven, or knitted tube that is formed of natural or prosthetic fibers extrinsic to the tensile load-bearing core might be used, so as to maintain all filaments exiting the coupled tissue in the service of load transfer. The braid material may be the same in composition and caliber as the coupling-forming, tissue-penetrating filaments, or it may be of a different permanent or absorbable material.

In still another alternative embodiment, the fiber materials may be chosen to increase hydrophilic behavior of their surfaces, and thus reduce any induction of autoimmune phenomena by a large hydrophobic surface. Material choice may include use of a strong biocompatible hydrophilic material such as very fine glass (silicon dioxide) fibers for construction. Alternatively, hydrophilic materials may be surface-deposited on a base fiber material that is chosen for mechanical properties of strength and flexibility.

Fiber bundles might be impregnated, in another embodiment, with a readily absorbable and/or soluble, flexible material to render it compact and capable of sliding through tissue with low friction, while having the multifilamentous nature restored upon dissolution or absorption of the impregnating material. The individual bundles of filaments of the device may be made compact by encasing the several-thousand fiber bundle in an absorbable material such as gelatin, glucose, or any other substance. It is desirable for the resulting strand to be as slender as possible (cross-sectional area near the combined fiber cross-sections times a reasonable packing factor), to be flexible and smooth at the time of surgical placement, but to then, as soon as is practical, to be free of any extrinsic bonding material so that fibers may disperse in tissue, tissue may penetrate between them, or both.

Nonlimiting examples of such possible impregnating materials include gelatin, with or without controlled cross-linking by chemical (e.g., adding dilute solutions of glutaraldehyde, beta propiolactone, or formaldehyde)), thermal, or radiation exposure. One specific method includes preparation of an aqueous solution of gelatin and saturating the bundle of fibers in it. A cross-linking agent is added to the solution shortly before saturation. The bundle is then drawn into an enveloping tube such as silicone rubber, and the solution allowed to gel. Then the tube is removed and the encased bundle allowed to dry. The resulting structure is relatively rigid, but upon soaking in water or an aqueous solution, it first softens and becomes flexible, and then the bonding matrix dissolves. Controlling factors may adjust the degree of cross-linking and thus both the time required to achieve flexibility and the working time between softening/flexibility and freeing of fibers to disperse.

For the example given, partially cross-linked gelatin, with cross-linking achieved by a solution additive such as formalin, there is a spectrum from near immediate dispersion to permanent insolubility. Between these extremes, the gelatin may be titrated. Means of titration include choice and concentration of cross-linking agent, time of addition before saturation of bundle, time to achieving and time left in gel state before drying, temperature of all processes, and humidity or other conditions while drying. Alternative methods to achieve titrated solubility include other cross-linking means such as immersion of a bundle prepared without cross-linking agent, whether still ungelled, gelled, or dried, into a liquid cross-linking agent so as to concentrate retarded solubility in the outer layers, exposing a similar bundle to a cross-linking gas, heat, ionizing or non-ionizing radiation, or other cross-linking methods commonly known in industry.

Other examples of impregnating materials include glycerine, a protein adhesive such as casein or casein derivatives, or any nontoxic absorbable polymer, or mix of polymers, natural or synthetic, of suitable physical properties and time course of dissolution in vivo.

In still another alternative embodiment, other features may be incorporated in the invention for anchoring to and in bone. For example, the needles used in soft-tissue couplers may be adapted for manually driving through the cortex of a bone by means well known in surgical applications (e.g., with stainless steel wire sutures commonly used for closure of the sternum [breastbone] in heart operations). As an alternative, holes may be drilled through one or more cortical faces of a bone and fibers pulled through by a needle that is not necessarily sharpened. As a further alternative, an absorbable filler may be formulated so as to render the end of a needle-less bundle of fibers (ranging from a few hundred to several thousand fibers) relatively rigid, so that it may be inserted through one drilled hole in the cortex of a bone into the cancellous bone and marrow from which it may or may not exit through a second cortical hole. Connecting fiber connectors of the types described above and in prior patent and patent applications to a metal or polymer bone anchor of any type might also be used. Special means for distributing stress at the bone surface might also be used. An intrinsic potential limitation of any flexible coupling (such as these fiber coupling devices) to a rigid structure (such as bone) is stress concentration, so that cyclic bending over a few days to several years may cause fatigue failure of at least a part of the fibers, with fragmentation. This may be ameliorated in various different ways in the invention. Specifically, drilling a larger hole in the bone cortex than needed for the fibers and allowing natural healing process to fill the space about the fibers with first blood coagulation products, principally fibrin, may be used. Then, it is replaced over time with collagenous tissue, which is softer (i.e., has a lower elastic modulus than) bone.

Referring to FIGS. 7A and 7B, these Figures illustrate a section of a bone 86 coupled in accordance with the present invention. Specifically, referring to FIG. 7A, a section of bone 86 includes cortical 88 and cancellous 90 portions into which a hole 92 has been drilled. A fiber bundle 94 has been inserted into the hole 92 by means of a needle or some other insertion mechanism (not shown). FIG. 7B illustrates a similar section of bone 86 after biologic processes have generated loose connective tissue 96 to ingrow and surround the fiber bundle 94.

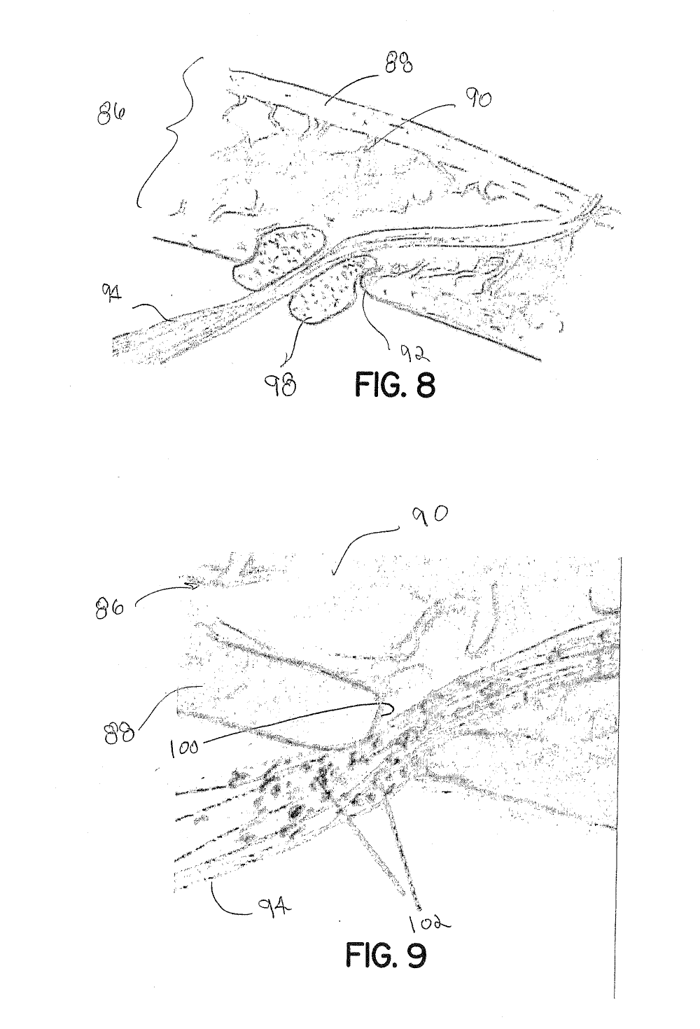

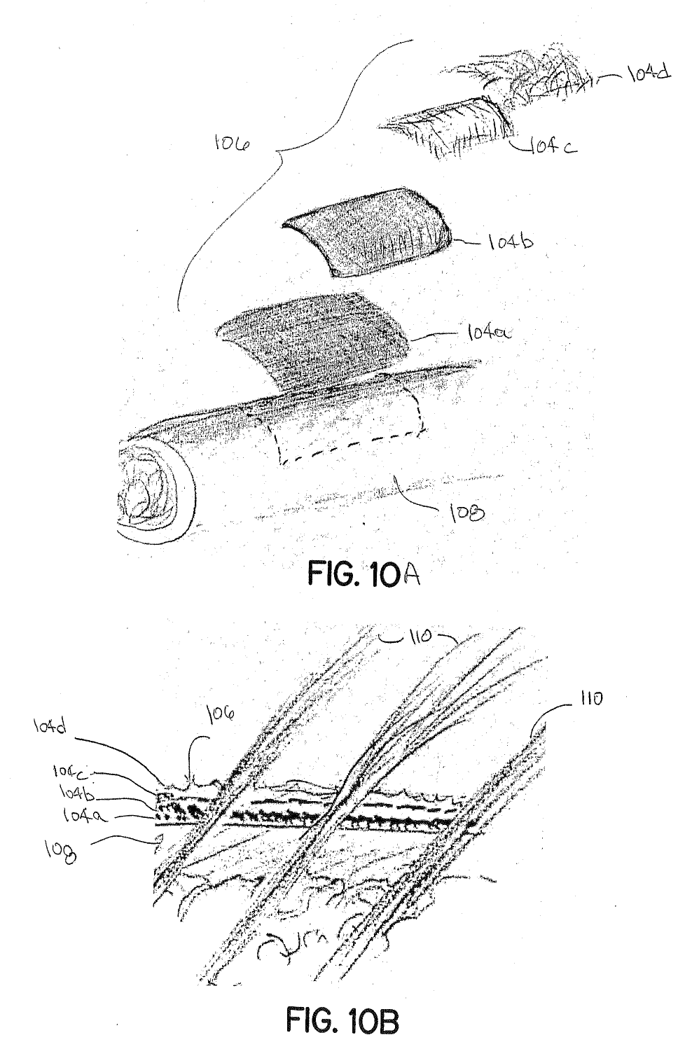

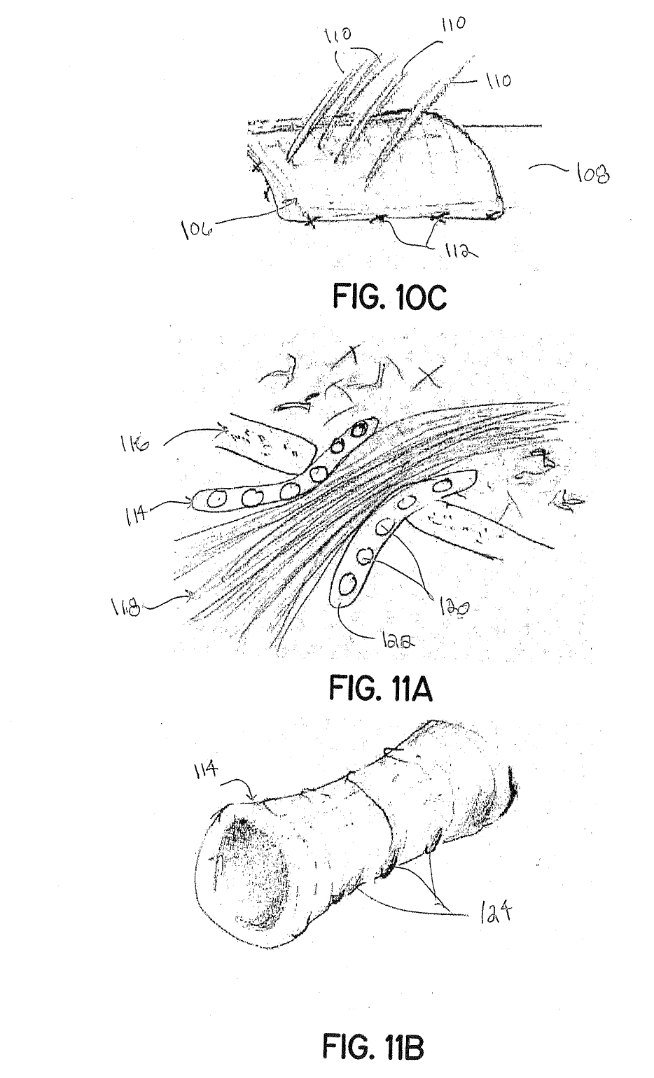

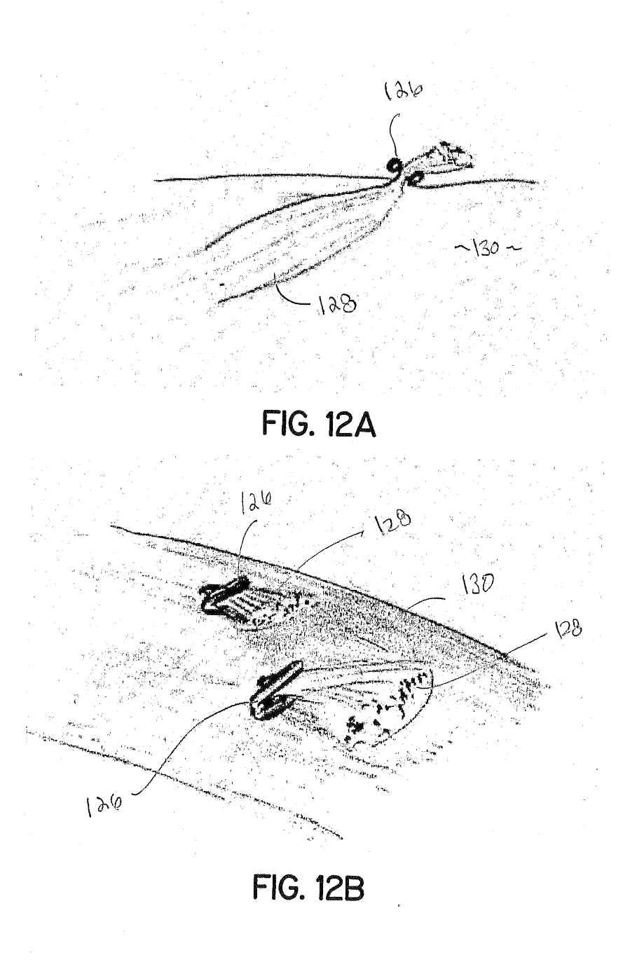

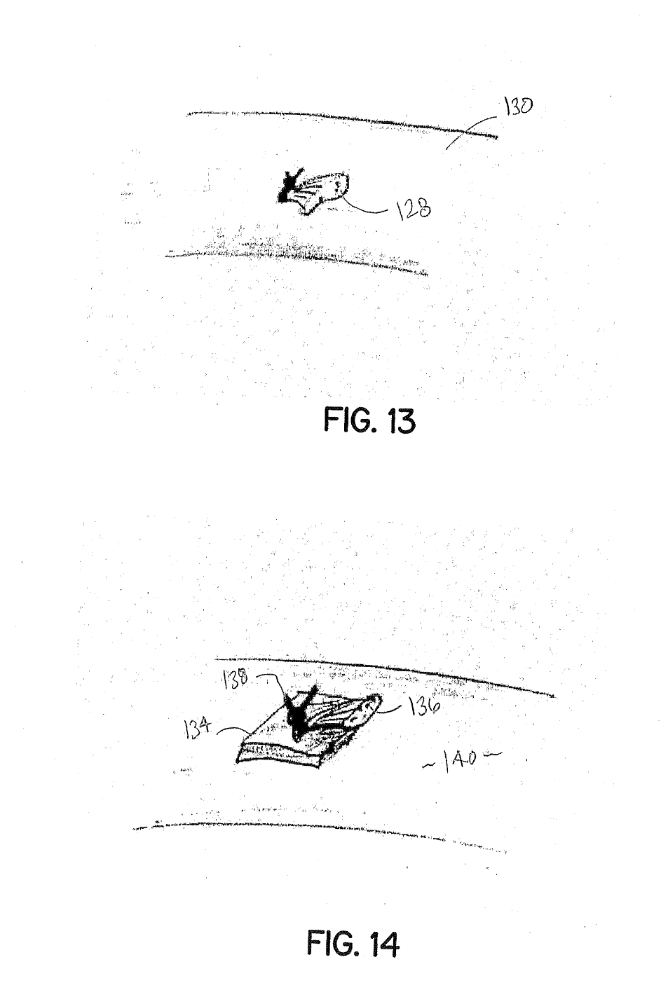

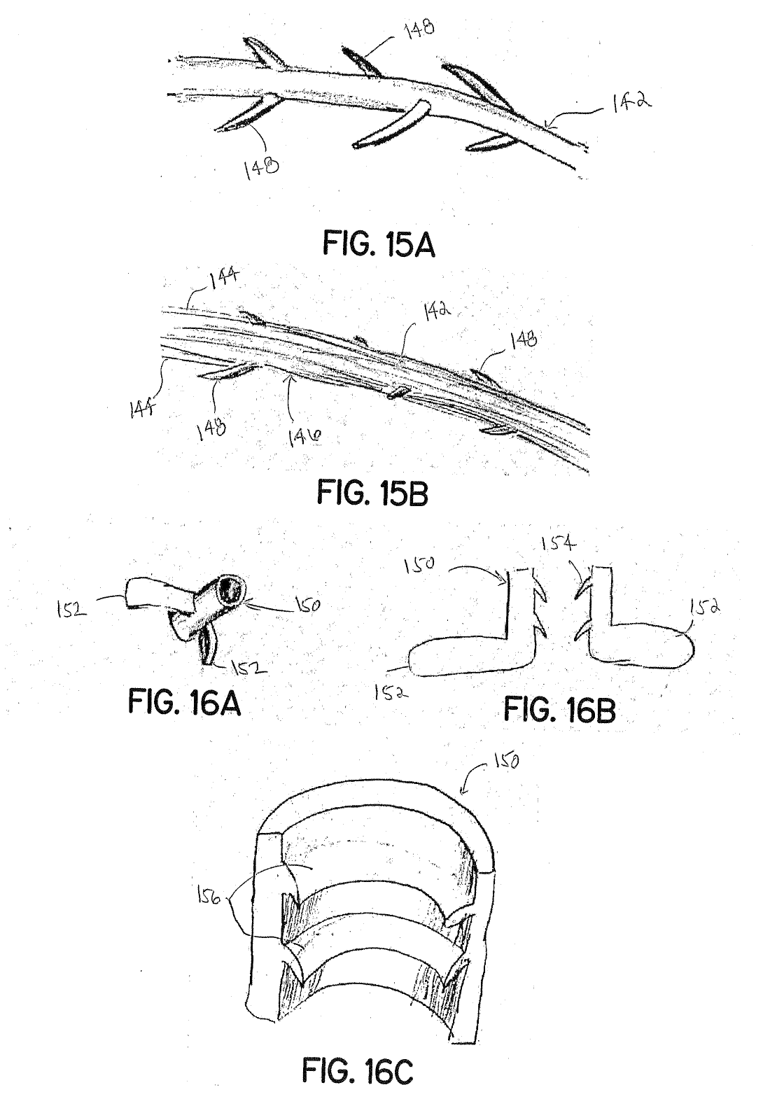

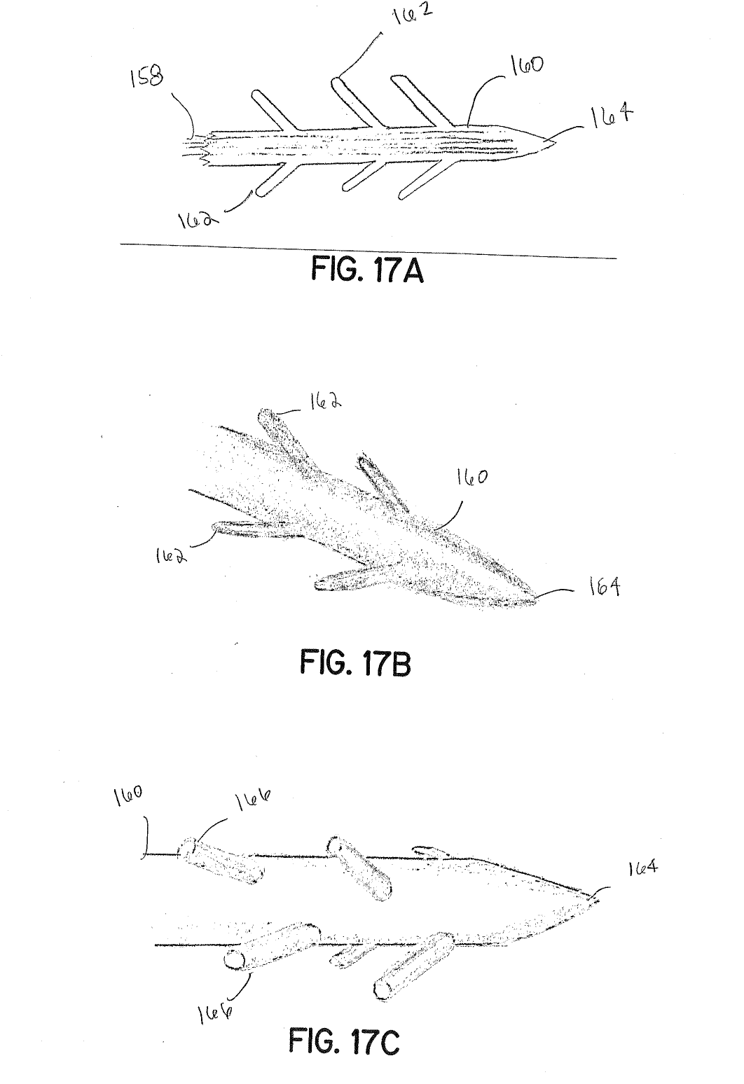

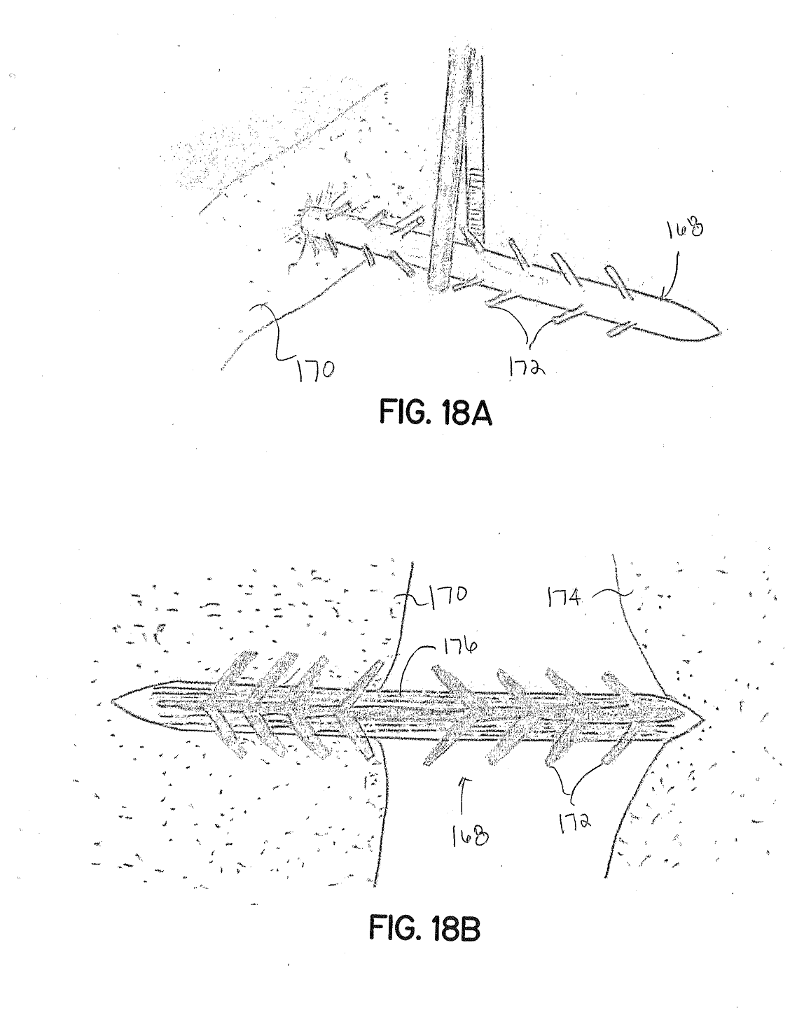

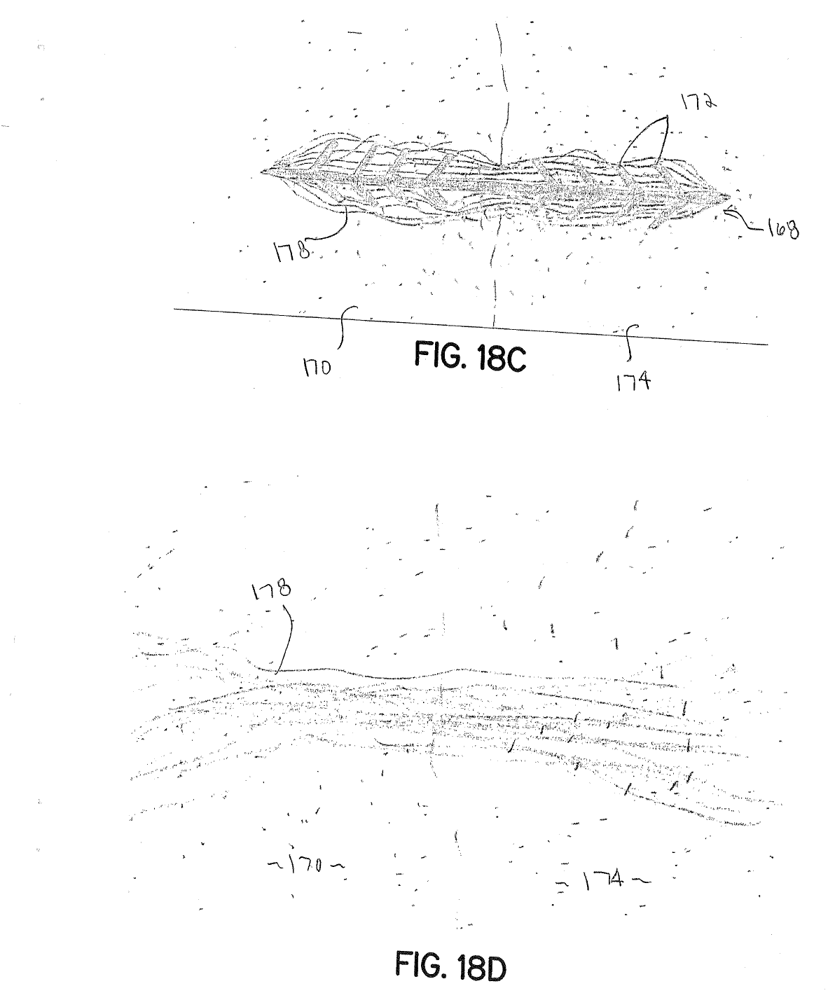

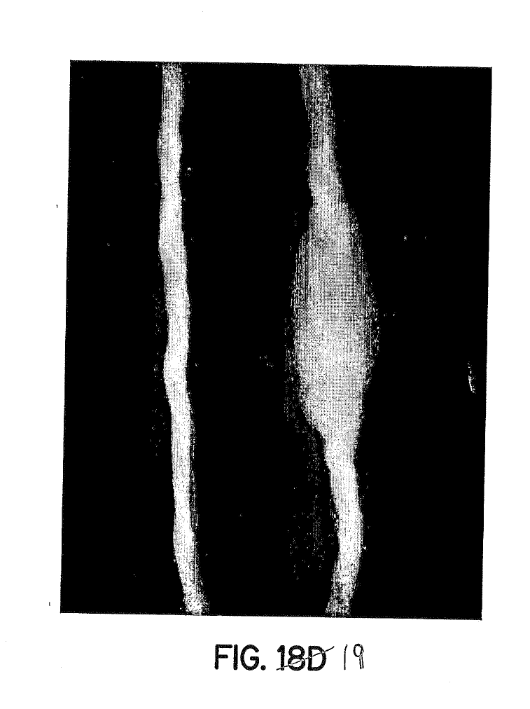

As shown in FIG. 8, a larger hole 92 may be drilled in the bone cortex 88, increasing the diameter of the fiber bundle 94 in the cortex-penetrating segment by locally dispersing fibers in an elastomeric ‘plug’ 98 designed to fill the hole 92. This elastomeric plug 98 may be made of any commonly used non-absorbable elastomer such as a polyurethane or a silicone rubber or a soft absorbable material such as partially cross-linked gelatin foam. In the case of an absorbable elastomeric plug 98, the intended end result is collagenous tissue, for a configuration similar to that of FIG. 7B.

In still another embodiment, materials known to stimulate bony absorption or osteolysis locally may be incorporated within the fiber bundle 94, at least within the segment intended to traverse the initial entry 100 through cortical bone 88. These materials may be adsorbed onto the surface of the fibers or included in an absorbable or nonabsorbable plug, such as plug 98. A nonlimiting example is very fine (a few microns) fragments of ultrahigh molecular weight polyethylene or other solid polymer which have been shown to stimulate foreign body giant cells that act as osteoclasts (bone-absorbing cells). While this is a unfavorable occurrence in the setting of a joint replacement where needed rigidity locally is lost, it would be favorable in the setting of fibrous coupling to bone, such as that shown in FIG. 9, where a transition to lesser rigidity at the exit is desired. Referring to FIG. 9, particulate material 102 is dispersed into the fiber bundle 94 for the purpose of inducing bone resorption or osteoclysis in the bone cortex 88. The osteoclysis-inducing agent may alternatively be a nonparticulate material absorbed onto or embedded in the fiber material from which it is subsequently eluded.

In yet another alternative embodiment, placement of a patch on the bone surface with graded stiffness across its thickness from very stiff at the interface with bone to very soft at the interface with surrounding soft tissue may be used. A nonlimiting example of a construction is several laminae of woven or knitted fabric, such as polyester or polytetraflurethyene or polyethylene, in which the bony interface may be a very tight weave, with subsequent interfaces progressing through a non-quite-as tight weave, with knits of progressively less tightness, to a soft-tissue contacting layer that is a very loose mesh.

Referring to FIG. 10A, an exploded view of layers 104a-d of a multi-laminate patch 106 is shown to be attached to the surface of bone 108, such as by means of an adhesive, sutures, or another suitable means. The layers 104a-d of the patch 106 are progressively stiffer as they progress toward the surface of the bone 108. Thus, layer 104a is stiffer than 104b, which is stiffer than 104c, etc. For example, the weave of each layer may become more tightly woven as the layers get closer to the bone. In accordance with this embodiment, FIG. 10B illustrates several fiber bundles 110 inserted through the multi-laminate patch 106 and into the bone 3. FIG. 10C is an external perspective view of several fiber bundles 110 entering patch 106. These may become, in various embodiments of the invention, organized together or inserted separately into the other tissue or the prosthetic structure to which the bone 108 is to be fixed. The patch 106 may be attached to the bone 108 or to its periosteal covering tissue by adhesives or by means of sutures 112.

In another alternative embodiment, as shown in FIGS. 11A and 11B, exiting fiber bundles are surrounded with a several-millimeter-long coil of metal or polymer, which may be of graduated and decreasing stiffness, and/or may be encased in a soft elastomer. It has the advantage of presenting a smooth surface to the fibers, expected to be far less abrasive and fraying than a bone margin without such protection. FIG. 11A illustrates a sectional view of a coil stress-distributing member 114, which surrounds a fiber bundle at the site of entry or exit of a hard tissue, such as a bone 116. Specifically, filaments or fiber bundle 118 is surrounded by the turns 120 of a polymer or metal coil, which may be encased in an elastomer 122. FIG. 11B is a perspective view of the coil stress-distributing member 114 before implantation. The outer surface may be configured to stabilize its position in bone or other hard tissues by phalanges or helical threads 124. These may or may not be coincident with the underlying helices or turns 120 of a reinforcing coil, as illustrated in FIG. 11A.

In another embodiment, means may be used to immediately stabilize the position of fibers within a tissue to allow time for development of a broad tissue-prosthetic interface. The fiber bundle will generally be cut a short distance beyond the stabilization structure (suture, patch, etc, as described below) following its application, with excess fiber bundle length removed. Various techniques and devices may be utilized for stabilization, such as tying adjacent fiber bundles, in groups of two or more, to each other as shown in FIG. 3B. Commercially available metal or polymer clips, which may or may not be absorbable, may be placed across the fiber bundle as it exits the lateral tissue surface. Nonlimiting examples are clips marketed by numerous venders for vessel ligation. Referring to FIG. 12A, a sectional view of a stabilizing clip 126 is shown on an exiting fiber bundle 128 near its exit from soft or hard tissue 130. FIG. 12B is a top perspective view of the stabilizing clip device 126, as utilized in FIG. 12A. Alternatively, and as shown in FIG. 13, an exiting fiber bundle 128 might be secured to the surface of the tissue 130 with an absorbable or nonabsorbable suture 132 near its exit point.

In another embodiment, a fiber bundle may be sutured to a prosthetic patch, with or without the fiber bundle having first traversed a puncture or slit in the patch. Nonlimiting examples are a ‘pledget’ of Teflon® felt, supplied and in common surgical use both precut to convenient sizes and in sheets to be cut as needed and small patches of autologous (the recipient's own) tissue recovered from the operative field (such as small rectangles or triangles of pericardium or muscle fascia). FIG. 14 shows a patch or pledget 134 to which an exiting fiber bundle 136 is anchored by a suture 138, resting on a tissue surface 140. The patch 134 itself may or may not be secured to the tissue surface 140 by the same suture(s), separate suture(s), or other means, such as an adhesive.

In another embodiment, included within the fiber bundle is one or more suture strands, generally but not necessarily absorbable and of a different material than the material of which primary fibers in the bundle, which have flexible side extensions or barbs. Referring to FIG. 15A, a barbed strand 142 is shown that is to be nested along with the filaments 144 of a fiber bundle 146 to provide physical stability to its placement during the interval of tissue ingrowth among the filaments 144. Barbs 148 extend from the strand 142, with one or more of the barbed strands included in the fiber bundle 146. FIG. 15B illustrates a barbed strand 142 surrounded by the filaments 144 of the fiber bundle 146.

As another alternative means of stabilization, adhesive compounds, generally absorbable, that are activated by presence of tissue, tissue fluid, or by an active intervention at time of implantation (nonlimiting examples being physical application of an activating agent or exposure to ultraviolet light or heat) that cause fixation of at least one region of the fiber bundle to tissue for a sufficient period to allow ingrowth and stable fixation, may be used.

Alternatively, a flanged ‘slider’, placed on each fiber bundle after tissue penetration may be used. This is a generally tubular structure with a channel or lumen through which the fiber bundle is advanced, and has one or more flanges or wings on its outer surface that exert retaining force against the tissue surface from which the fiber bundle exits. It also has ratcheting structures on the inner surface of the channel that readily allow the fiber bundle to be advanced through it but wedge to exert substantial resistance to backward movement. Nonlimiting examples are rod-like barbs and web-like fins. FIG. 16A illustrates a slider 150 with multiple flanges 152 to exert a retaining force against the tissue surface (not shown). For example, FIG. 16B illustrates a cross-sectional view of slider 150 incorporating a ratcheting structure in the form of barbs 154. FIG. 16C also illustrates a slider 150 with a ratcheting structure in the form of circumferential flanges 156.

In another embodiment, shown in FIGS. 17A, 17B, and 17C, a fiber bundle 158 is impregnated by a rigid absorbable filler 160. The filler material has barb-like extensions 162 that are molded and configured to extend laterally into and anchor to surrounding tissue (not shown). The filler material may also have a pointed tip 164 which is either molded or machined onto it after hardening. FIGS. 17A, and 17B, show a longitudinal section and perspective views of such a device in which extensions 162 and the remainder of the filler 160 are cast of the same material. FIG. 17C shows a variation in which cut rod-like segments 166 of the same or another absorbable material are embedded in the filler 160 prior to its hardening.

Yet another embodiment, similar to this, is illustrated by FIG. 18. Here a central rigid core of an absorbable material has barb-like extensions. Fibers of the bundle are packed around the central core and fused to it by an absorbable filler which will generally have a faster rate of absorption, degradation, or dissolution than the central rigid core, A nonlimiting example is the double-ended rigid coupler 168 shown in perspective view in FIG. 18A, being inserted into a first tissue 170 to be joined. The coupler 168 illustrated has protruding lateral absorbable barbs 172 that such a double-ended coupler will generally, but not necessarily, include. FIG. 18B shows the coupler 168 in cross section as a second tissue 174 is brought adjacent to first tissue 170 and the other end of coupler 168 is pierced into that second tissue 174. There is an absorbable bonder or filler 176 present. FIG. 18C shows, in cross section, the coupler 168 after absorbable bonder or filler 176 has been absorbed, allowing tissue ingrowth into the fiber bundle 178. FIG. 18D shows, in cross section, the coupler 168 after absorbtion of the barbed strands, leaving only the fiber bundle 178 holding the two tissues 170, 174 which have locally grown between and dispersed the fibers.

In still another embodiment, the physical configuration of the filaments in a bundle is altered, such that they are more dispersed relative to each other, with the intent of facilitating tissue in growth. This is accomplished by applying longitudinal force sufficient to produce between 0 and 20% strain. This causes a differential deformation across the diameter of fibers which appears to be random, and in turn causes the bundle of filaments, when under less tension, to have randomly assorted curvatures across the various component filaments and various segments of the same filament. In FIG. 19, the bundle on the right (otherwise identical to the left bundle) has been subject to around 5-10 pounds (i.e., 45-90 Newtons) of longitudinal force for a duration of about ten seconds, and released. The deformation or spreading of the fiber bundle on the right resulted.

While the present invention has been illustrated by the description of the embodiments thereof, and while the embodiments have been described in considerable detail, it is not the intention of the applicant to restrict or in any way limit the scope of the appended claims to such detail. Additional advantages and modifications will readily appear to those skilled in the art. Therefore, the invention in its broader aspects is not limited to the specific details representative apparatus and method, and illustrative examples shown and described. Accordingly, departures may be made from such details without departure from the spirit or scope of applicant's general inventive concept.

What is claimed:

1. A tissue coupling device comprising:
a plurality of slender fibers extending in generally parallel fashion from within a first tissue substance to within a second tissue substance and across a tissue boundary, said plurality of fibers configured to provide a scaffolding for capturing proliferating cells of said first and second tissue substances to envelope said fibers and provide mechanical coupling across said tissue boundary.

2. The device of claim 1, wherein said fibers are made from a metal selected from the group consisting of stainless steel, titanium, titanium alloy, nickel-titanium alloy, and cobalt-chromium alloy.

3. The device of claim 1, wherein said fibers are made from a synthetic polymer selected from the group consisting of polyester, polyethylene, polypropylene, a liquid crystal polymer, an aramid, polyglycolic acid, and polyurethane.

4. The device of claim 1, wherein said fibers are made from a natural polymer selected from the group consisting of collagen, elastin, fibronectin, and gelatin.

5. The device of claim 1, wherein said fibers are made from a ceramic of silicon dioxide.

6. The device of claim 1, wherein said surface of said fibers is roughened so as to increase the coefficient of friction of said fibers or to increase tissue adhesion with said fibers.

7. The device of claim 1, further comprising:
a rigid elongated member having an insertion end and coupled with said plurality of fibers.

8. The device of claim 7, wherein said rigid member is formed from a plurality of said fibers bonded to form said rigid member.

9. The device of claim 1, further comprising:
a stabilizing device selected from the group consisting of a surgical ligating clip, a suture, a patch, a slide, a barbed tube, a barbed strand, and barbed fibers, said stabilizing device coupled to said plurality of fibers adjacent an exit of said plurality of fibers from the tissue and adapted to allow time for tissue ingrowth and adhesion after placement of said plurality of fibers in the tissue.

10. The device of claim 1, further comprising
an absorbable material encasing said plurality of fibers.

11. The device of claim 10, wherein said absorbable material is selected from the group consisting of a protein, cross-linked protein, and gelatin.

12. The device of claim 10, wherein said absorbable material comprises an thin absorbable sheath adapted to enclose and compact said plurality of fibers for easing the passage of said plurality of fibers into the tissue.

13. A method for tissue coupling, comprising:
positioning a first tissue substance and a second tissue substance proximate each other to form a tissue boundary; and
extending a plurality of fibers from within the first tissue substance and into the second tissue substance and across the tissue boundary to couple the first tissue substance with the second tissue substance, wherein the plurality of fibers are generally oriented in planes parallel to a predominant direction of known or potential tissue disrupting forces.

14. The method of claim 13, wherein the plurality of fibers provide a scaffolding for capturing proliferating cells of the first and second tissue substances to envelope the fibers and provide mechanical coupling across the tissue boundary.

15. The method of claim 13, further comprising:
roughening a surface of the plurality of fibers by a method selected from the group consisting of mechanical abrasion, sintering, chemical etching, bombardment by subatomic particles, and irradiation.

16. The method of claim 13, further comprising:
impregnating the plurality of fibers with an absorbable material, said absorbable material including a cross-linked protein made by a method selected from the group consisting of chemical treatment, thermal treatment, and radiation treatment.

17. The method of claim 13, further comprising:
treating the plurality of fibers by depositing at least one layer of a material on the fibers through a process selected from the group consisting of electrostatic self-assembly, solution casting, vapor deposition, and particle sputtering.

18. The method of claim 13 further comprising:
forming a pointed elongate rigid member by bonding at least a portion of the plurality of fibers into a single unit, the bonding being achieved through a process selected from the group consisting of thermal fusing, solvent sealing, and using an impregnating bonding agent,

19. The method of claim 13, wherein at least one of the first and second tissue substances is skeletal muscle, uterine muscle, smooth muscle, cardiac muscle, loose connective tissue, dense connective tissue, fat, brain, liver, spleen, exocrine gland, endocrine gland, bone, and tooth.

20. The method of claim 13 as applied to a medical procedure selected from the group consisting of re-approximation of previously joined tissue after purposeful incision, re-approximation of previously joined tissue after traumatic disruptions, excision of diseased tissue, repair of uteri after fibromyotomy or caesarean section, repair of cardiac chambers, and the reimplantation of teeth.
